(19) United States
(12) Patent Application Publication
(10) Pub. No.: US 2007/0251028 A1
(43) Pub. Date: Nov. 1, 2007

(54) Dyeing composition comprising at least one electrophilic monomer, one hydrophilic dye and one liquid organic solvent

(76) Inventors: Henri Samain, Bievres (FR); Leila Hercouet, Neuilly Plaisance (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,924

(22) Filed: Apr. 13, 2007

Related U.S. Application Data
(60) Provisional application No. 60/802,107, filed on May 22, 2006.

(30) Foreign Application Priority Data
Apr. 13, 2006 (FR) 0603317

(51) Int. Cl. A61K 8/00

(52) U.S. Cl. 8/405

(57) ABSTRACT

The present disclosure relates to a composition for dyeing keratinous substances, including keratinous fibers such as the hair, comprising at least one electrophilic monomer, at least one hydrophilic dye and at least one liquid organic solvent. The present disclosure also relates to a process for dyeing keratinous substances and to the use of the composition in dyeing keratinous substances. The composition of the present disclosure improves the resistance of the coloring of keratinous substances to external assaults while exhibiting a broad range of hues and without denaturing the keratinous substances.

This application claims benefit of U.S. Provisional Application No. 60/802,107, filed May 22, 2006, the contents of which are incorporated herein by reference. This application also claims benefit of priority under 35 U.S.C. § 119 to French Patent Application No. FR 06/03317, filed Apr. 13, 2006, the contents of which are also incorporated herein by reference.

The present disclosure relates to a composition for dyeing keratinous substances, for example, keratinous fibers such as the hair, comprising at least one electrophilic monomer, at least one hydrophilic dye and at least one liquid organic solvent. The present disclosure also relates to a process for dyeing keratinous substances and to the use of the aforementioned composition for dyeing keratinous substances.

Scientific research has been carried out for some time now on modifying the color of keratinous substances, e.g., keratinous fibers, and in particular on masking white fibers.

In the field of dyeing keratinous fibers, it is already known to dye keratinous fibers by various techniques, starting from direct dyes or pigments for non-permanent colorings or from dye precursors for permanent colorings.

Oxidation dyeing comprises applying, to keratinous fibers, a composition comprising dye precursors, such as oxidation bases and couplers. These precursors, under the action of an oxidizing agent, form one or more colored entities in the individual hair fibers. The variety of the molecules employed as oxidation bases and couplers makes it possible to obtain a rich palette of colors, and for the colorings resulting therefrom to be permanent, powerful and/or resistant to external agents, such as light, bad weather, washing operations, perspiration and rubbing.

However, repeated dyeing with oxidizing agents may damage the cosmetic and mechanical properties of the fibers, which may result in difficult styling or hair shaping.

The use of pigments for dyeing keratinous materials, such as keratin fibers, is also known. Use of pigment at the surface of keratinous fibers generally makes it possible to obtain visible colorings on dark hair, since the surface pigment masks the natural color of the fiber. This method of dyeing keratinous fibers is described, for example, in French Patent Application FR 2 741 530, which recommends the use, for the dyeing of keratinous fibers, of a composition comprising at least one dispersion of particles of film-forming polymer comprising at least one acid functional group and at least one pigment dispersed in the continuous phase of the dispersion.

The colorings obtained by this dyeing method, however, may exhibit the disadvantage of having an unsatisfactory resistance to shampooing operations.

It is further known to dye keratinous fibers such as the hair with dyeing compositions comprising direct dyes. This technique is commonly referred to as “non-permanent dyeing” or “direct dyeing”.

Direct dyes are colored and coloring molecules which exhibit a degree of affinity for keratinous fibers. They are applied to keratinous fibers for a time necessary for the desired coloring to be obtained and are then rinsed. The colorings which result therefrom are often chromatic colorings which, however, are temporary or semi permanent as the nature of the interactions that bind direct dyes to the keratinous fibers and their desorption from the surface and/or from the core of the fiber are responsible for their low dyeing power and for their poor resistance to washing operations or to perspiration.

French Patent Application FR 2 833 489 discloses compositions for the treatment of the hair that are derived from electrophilic monomers capable of polymerizing anionically directly on the hair. These monomers, after polymerization, make it possible to obtain perfectly sheathed hairs. However, the sheathing obtained from electrophilic monomers may not exhibit satisfactory strength with respect to the various assaults to which hair may be subjected, such as shampooing operations, sebum, rubbing, light, bad weather, sweat and permanent deformations.

Thus, there is a desire to develop novel direct dyeing compositions for the purpose of obtaining varied hues, in particular pastel hues, which exhibit good resistance, in particular to external agents, such as light, shampoos or sweat, and which furthermore make it possible to keep the keratinous fibers intact.

Specifically, there is a desire to develop dyeing compositions which make it possible to obtain colorings exhibiting a resistance similar to that of oxidation dyeing, but without one or more of the disadvantages related to the presence of an oxidizing agent.

The present disclosure relates to dyeing compositions comprising, in a medium suitable for dyeing, at least one electrophilic monomer, at least one hydrophilic direct dye having a logP of less than or equal to 2, and at least one liquid organic solvent. These dyeing compositions make it possible to enhance the persistence of a dye on a keratinous substance by virtue of the combination of the dye with at least one polymerizable electrophilic monomer.

Application of the compositions of the present disclosure to keratinous fibers such as the hair results in the formation of a sheathing at the surface of individual hairs that may be persistent towards external assaults, such as sebum, light or shampooing. This sheathing is homogeneous and smooth and possesses excellent adhesion to the hair.

Further, the at least one electrophilic monomer forms a polymer that coats the hydrophilic dye, resulting in a colored sheathing that may be persistent towards external agents.

In addition, individual hairs treated with the composition of the present disclosure remain separate from one another, and are easily styled.

The compositions according to the present disclosure also make it possible to obtain hues varying from pastel to intense colorings, while remaining resistant to one or more of the various assaults to which hair may be subjected, such as shampooing operations, rubbing, light, bad weather, sweat and permanent deformations. For example, keratinous materials treated with the compositions of the present disclosure may exhibit colorings that exhibit substantially improved resistance to repeated shampooing operations.

The present disclosure also relates to a process for dyeing keratinous substances. This process comprises applying the compositions of the present disclosure to keratinous substances, for example keratinous fibers such as the hair.

The present disclosure also relates to a multi-compartment kit comprising, in one compartment, a composition comprising at least one electrophilic monomer and at least one direct dye having a logP of less than or equal to 2 and, in another compartment, a second composition which comprises at least one nucleophilic agent. The at least one liquid organic solvent may be present in either of the compartments.

In another embodiment, the kit comprises, in one compartment, a composition comprising at least one electrophilic monomer and, in another compartment, a second composition comprising at least one direct dye having a logP of less than or equal to 2, it being understood that the at least one organic liquid solvent can be found in either of the compartments. In one embodiment, the solvent is present in the compartment comprising the electrophilic monomer.

The at least one electrophilic monomer of the present disclosure may, for example, be chosen from:

the derivatives benzylidenemalononitrile (A1), 2-(4-chlorobenzylidene)malononitrile (A2), ethyl 2-cyano-3-phenylacrylate (B1) or ethyl 2-cyano-3-(4-chlorophenyl)acrylate (B2), described in Sayyah, J. Polymer Research, 2000, p 97;

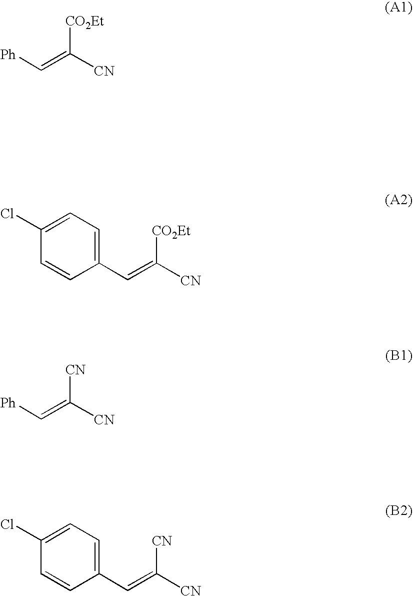

methylidenemalonate derivatives, including, but not limited to:
diethyl 2-methylenemalonate (C1), described by Hopff, Makromoleculare Chemie, 1961, p 95, De Keyser, J. Pharm. Sci., 1991, p 67 and Klemarczyk, Polymer, 1998, p 173; 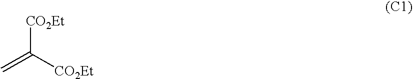
ethyl 2-ethoxycarbonylmethyloxycarbonylacrylate (D1), described by Breton, Biomaterials, 1998, p 271 and Couvreur, Pharmaceutical Research, 1994, p 1270;

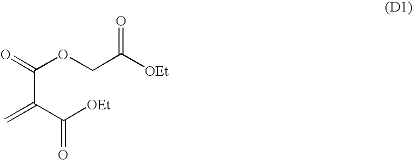

Itaconate and itaconimide derivatives, including, but not limited to:
dimethyl itaconate (E1), described by Bachrach, European Polymer Journal, 1976, p 563; 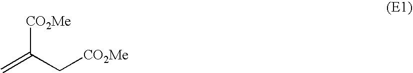
N-butylitaconimide (F), N-(4-tolyl)itaconimide (G), N-(2-ethylphenyl)itaconimide (H) and N-(2,6-diethylphenyl)itaconimide (I), described by Wanatabe, J. Polymer Science: Part A: Polymer Chemistry, 1994, p 2073;

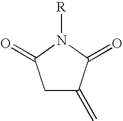

R=Bu (F), 4-tolyl (G), 2-ethylphenyl (H), 2,6-diethylphenyl (I);

the derivatives methyl α-(methylsulphonyl)acrylate (K), ethyl α-(methylsulphonyl)acrylate (L), methyl α-(tert-butylsulphonyl)acrylate (M), tert-butyl α-(methylsulphonyl)acrylate (N) and tert-butyl α-(tert-butylsulphonyl)acrylate (O), described by Gipstein, J. Org. Chem., 1980, p 1486, and the derivatives 1,1-bis(methylsulphonyl)ethylene (P), 1-acetyl-1-(methylsulphonyl)ethylene (Q), methyl α-(methylsulphonyl)vinylsulphonate (R) and α-(methylsulphonyl)acrylonitrile (S), described by Shearer, U.S. Pat. No. 2,748,050;

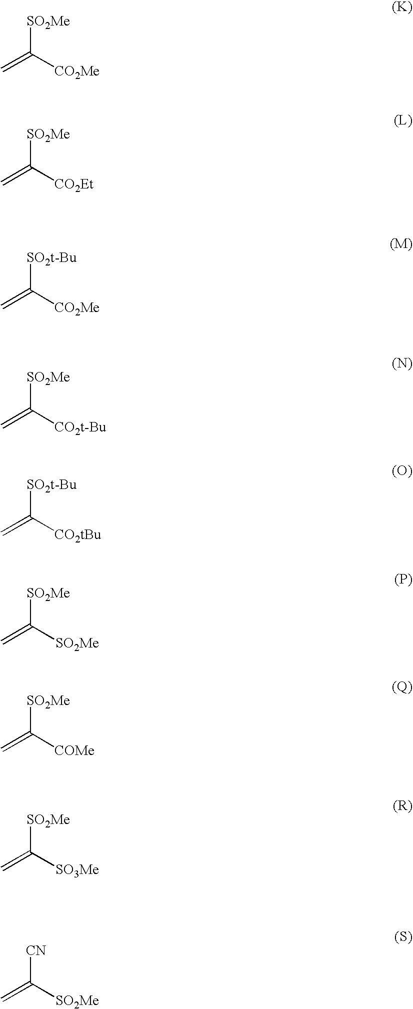

the derivatives methyl vinyl sulphone (T) and phenyl vinyl sulphone (U), described by Boor, J. Polymer Science, 1971, p 249;

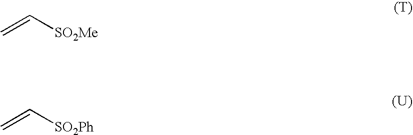

the derivative phenyl vinyl sulphoxide (V), described by Kanga, Polymer Preprints (ACS, Division of Polymer Chemistry), 1987, p 322;

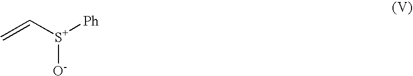

the derivative 3-methyl-N-(phenylsulphonyl)-1-aza-1,3-butadiene (W), described by Bonner, Polymer Bulletin, 1992, p 517;

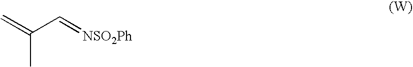

acrylate and acrylamide derivatives, such as:
N-propyl-N-(3-triisopropoxysilylpropyl)acrylamide (X) and N-propyl-N-(3-triethoxysilylpropyl)acrylamide (Y), described by Kobayashi, Journal of Polymer Science, Part A: Polymer Chemistry, 2005, p 2754; 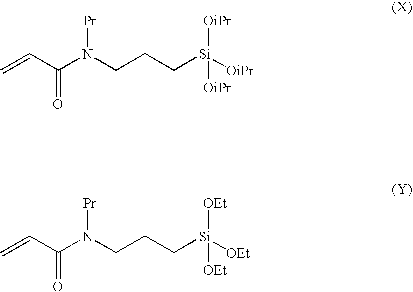
2-hydroxyethyl acrylate (Z) and 2-hydroxyethyl methacrylate (AA), described by Rozenberg, International Journal of Plastics Technology, 2003, p 17; 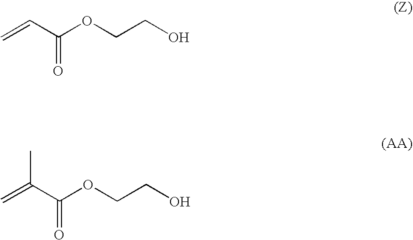
N-butyl acrylate (AB), described by Schmitt, Macromolecules, 2001, p 2115;
tert-butyl acrylate (AC), described by Ishizone, Macromolecules, 1999, p 955;

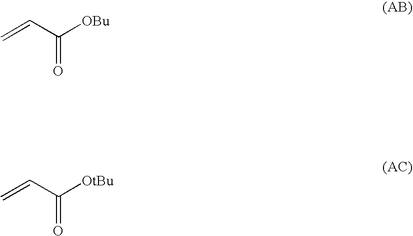

The at least one electrophilic monomer of the present disclosure may be cyclic or linear and comprise at least one electron-withdrawing group. When the monomer is cyclic, the electron-withdrawing group, in at least one embodiment, is exocyclic, i.e., it does not form an integral part of the cyclic structure of the monomer.

According to at least one embodiment of the present disclosure, the at least one electrophilic monomer comprises at least two electron-withdrawing groups.

As examples of monomers having at least two electron-withdrawing groups, non-limiting mention may be made of the monomers of formula (A):

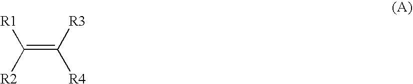

wherein:

R1 and R2 are each chosen, independently of one another, from groups having little to no electron-withdrawing effect (with little to no inductive withdrawing effect) such as:
hydrogen,
saturated or unsaturated, linear, branched or cyclic hydrocarbon groups, comprising, in at least one embodiment, from 1 to 20 carbon atoms, such as from 1 to 10 carbon atoms, optionally comprising at least one atom chosen from nitrogen, oxygen and sulphur atoms and optionally substituted by at least one group chosen from —OR, —COOR, —COR, —SR and halogen atoms,
modified or unmodified polyorganosiloxane residues,
polyoxyalkylene groups,

wherein, in at least one embodiment, R1 and R2 are both hydrogen atoms;

R3 and R4 are each chosen, independently of one another, from electron-withdrawing (or inductive-withdrawing) groups chosen, in at least one embodiment, from —N(R)₃⁺, —S(R)₂⁺, —SH₂⁺, —NH₃+, —NO₂, —SO₂R, —C≡N, —COOH, —COOR, —COSR, —CO(NH)₂, —CONHR, —CON(R)₂, —F, —Cl, —Br, —I, —OR, —COR, SH, —SR and —OH groups, linear or branched alkenyl groups, linear or branched alkynyl groups, C₁-C₄ mono- or polyfluoroalkyl groups, aryl groups, such as phenyl, and aryloxy groups, such as phenoxyloxy;
wherein R is chosen from: saturated or unsaturated, linear, branched or cyclic hydrocarbon groups comprising, in at least one embodiment, from 1 to 20 carbon atoms, for example from 1 to 10 carbon atoms, optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer which can be obtained by radical polymerization, by polycondensation or by ring opening, wherein R′ is a C₁-C₁₀ alkyl group.

As used herein, the terms “electron-withdrawing group” or “inductive-withdrawing group” mean any group that is more electronegative than carbon. Reference may be made to the work P R Wells, Prog. Phys. Org. Chem., Vol. 6, 111 (1968).

As used herein the term “group with little or no electron-withdrawing effect” means any group having an electronegativity less than or equal to that of carbon.

In at least one embodiment of the present disclosure, the alkenyl or alkynyl groups mentioned above have 2 to 20 carbon atoms, for example from 2 to 10 carbon atoms.

In at least one embodiment, the saturated or unsaturated, linear, branched or cyclic hydrocarbon groups referred to above comprise from 1 to 20 carbon atoms, and are chosen from linear or branched alkyl, alkenyl or alkynyl groups, including but not limited to methyl, ethyl, n-butyl, tert-butyl, isobutyl, pentyl, hexyl, octyl, butenyl or butynyl; cycloalkyl groups or aromatic groups.

As substituted hydrocarbon groups, non-limiting mention may be made of hydroxyalkyl or polyhaloalkyl groups.

As non-limiting examples of unmodified polyorganosiloxanes, mention may be made, in at least one embodiment, of polyalkylsiloxanes, such as polydimethylsiloxanes, polyarylsiloxanes, such as polyphenylsiloxanes, or polyarylalkylsiloxanes, such as polymethylphenylsiloxanes.

As non-limiting examples of modified polyorganosiloxanes, mention may be made, in at least one embodiment, of polydimethylsiloxanes comprising polyoxyalkylene and/or siloxy and/or silanol and/or amine and/or imine and/or fluoroalkyl groups.

Among polyoxyalkylene groups, mention may be made, for example, of polyoxyethylene groups and polyoxypropylene groups having, in at least one embodiment, 1 to 200 oxyalkylene units.

As non-limiting examples of mono- or polyfluoroalkyl groups, mention may be made of groups of the formula —(CH₂)ₙ—(CF₂)ₘ—CF₃ and —(CH₂)ₙ—(CF₂)ₘ—CHF₂ where n and m independently range from 1 to 20.

The R1 to R4 substituents may optionally be substituted by a group having a cosmetic activity. In at least one embodiment, groups having such cosmetic activity include those having coloring, antioxidizing, UV-screening and/or conditioning functions.

Mention may be made, as examples of groups having a coloring function, of azo, quinone, methine, cyanomethine and triarylmethane groups.

Mention may be made, as examples of groups having an antioxidizing functon, of groups of butylated hydroxyanisole (BHA), butylated hydroxytoluene (BHT) or vitamin E type.

As non-limiting examples of groups having a UV-screening function, mention may be made of benzophenone, cinnamate, benzoate, benzylidenecamphor and dibenzoylmethane groups.

As non-limiting examples of groups having a conditioning function, mention may be made of cationic groups and fatty ester groups.

Of the monomers mentioned above, non-limiting mention may be made of cyanoacrylate monomers of formula (B) and derivatives thereof:

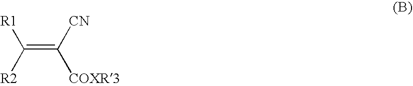

wherein:

X is chosen from NH, S and O,

R1 and R2 have the same meanings as in formula (A), and, in at least one embodiment, are both a hydrogen atom,

R′3 is a hydrogen atom or R as defined above for formula (A).

In at least one embodiment, X is an oxygen atom.

In another embodiment, R′3 is chosen from linear or branched alkyl radicals comprising from 6 to 10 carbon atoms and alkenyl radicals comprising from 2 to 10 carbon atoms.

As non-limiting examples of the cyanoacrylate monomers of formula (B), mention may be made of the following monomers:

a) monomers belonging to the family of the polyfluoroalkyl 2-cyanoacrylates, including, but not limited to: the 2,2,3,3-tetrafluoropropyl ester of 2-cyano-2-propenoic acid of formula (D):

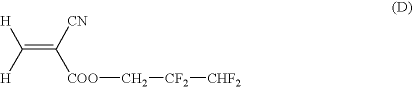

or the 2,2,2-trifluoroethyl ester of 2-cyano-2-propenoic acid of formula (E):

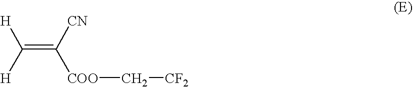

b) alkyl or alkoxyalkyl 2-cyanoacrylates of formula (C):

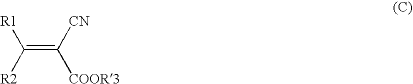

wherein:

R1 and R2 have the same meanings as above, and in at least one embodiment are both a hydrogen atom,

R′3 is chosen from C₁-C₁₀ alkyl, (C₁-C₄)alkoxy(C₁-C₁₀)alkyl, and C₂-C₁₀ alkenyl radicals.

In at least one embodiment, the cyanoacrylate monomers of formula (B) may be chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, isoamyl cyanoacrylate, allyl 2-cyanoacrylate and methoxypropyl 2-cyanoacrylate.

In another embodiment of the present disclosure, the cyanoacrylate monomers are chosen from monomers (b): alkyl or alkoxyalkyl 2-cyanoacrylates of formula (C) above. In a still further embodiment of the present disclosure, the cyanoacrylates are chosen from C₆-C₁₀ alkyl or C₂-C₁₀ alkenyl cyanoacrylates.

In yet a further embodiment, the monomers of formula (C) above are chosen from octyl cyanoacrylates of formula (F): 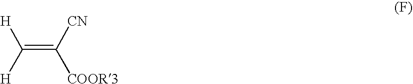
in which:
R′3 is chosen from:
   —(CH₂)₇—CH₃,
   —CH(CH₃)—(CH₂)₅—CH₃,
   —CH₂—CH(C₂H₅)—(CH₂)₃—CH₃,
   —(CH₂)₅—CH(CH₃)—CH₃,
   —(CH₂)₄—CH(C₂H₅)—CH₃.
      and mixtures thereof.

The monomers used in accordance with the present disclosure may be covalently attached to at least one support, such as a polymer, oligomer or dendrimer. The polymer or oligomer may be linear, branched, of a comb structure or of a block structure. The distribution of the monomers of the present disclosure over the polymer, oligomer or dendrimer structure can be random, in a terminal position, or in the form of blocks.

The electrophilic monomers may be synthesized according to known methods described in the art. For example, the cyanoacrylate monomers may be synthesized according to the disclosure of U.S. Pat. Nos. 3,527,224, 3,591,767, 3,667,472, 3,995,641, 4,035,334 and 4,650,826.

In the composition of the present disclosure, the electrophilic monomer is present, in at least one embodiment, in an amount ranging from 0.1 to 99.9% by weight, relative to the total weight of the composition, for example, from 1 to 50% by weight.

For the purpose of the present disclosure, the electrophilic monomers are monomers that are capable of polymerizing anionically in the presence of a nucleophilic agent.

As used herein, the term, “anionic polymerization,” means the mechanism defined in the work, “Advanced Organic Chemistry”, third edition, by Jerry March, at pages 151 to 161.

The nucleophilic agents able to initiate the anionic polymerization are systems known per se that are capable of generating a carbanion on contact with a nucleophilic agent, such as the hydroxyl ions present in water. As used herein, the term “carbanion” means the chemical entities defined in “Advanced Organic Chemistry”, third edition, by Jerry March, at page 141.

The nucleophilic agents can be applied independently of the composition of the present disclosure, and may be added to the composition of the present disclosure at the time of use.

Nucleophilic agents are molecular compounds, oligomers, dendrimers or polymers having nucleophilic functional groups. As non-limiting examples of nucleophilic functional groups suitable for use according to the present disclosure, mention may be made of the following functional groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, H₂O, wherein Ph is a phenyl group, Ar is an aryl group and R is a C₁-C₁₀ alkyl group.

In at least one embodiment of the present disclosure, the nucleophilic agent is water.

As used herein, the value of logP represents the coefficient of partition of the dye between octanol and water. The value of logP may be calculated according to the method described in the paper by Meylan and Howard, “Atom/fragment contribution method for estimating octanol-water partition coefficient”, J. Pharm. Sci., 1995, 84, p 83-92. This value may also be calculated using commercially available software, which determine the value of logP as a function of the structure of a molecule. As a non-limiting example of such software, mention may be made of the Epiwin software of the EPA (United States).

The direct dyes that are suitable for use in the composition of the present disclosure are known hydrophilic dyes exhibiting a logP value of less than or equal to 2.

In at least one embodiment of the present disclosure, logP is strictly less than 2.

Non-limiting examples of direct dyes that can be used according to the present disclosure include, but are not limited to neutral, acidic or cationic nitrobenzene direct dyes; neutral, acidic or cationic azo direct dyes; neutral, acidic or cationic quinone and in particular anthraquinone direct dyes; azine direct dyes; triarylmethane direct dyes; indoamine direct dyes and natural direct dyes.

The direct dyes carry at least two cationic or anionic functional groups.

The hydrophilic dyes of the present disclosure are chosen, for example, in such a way so as to be dissolved or dispersed in the composition of the present disclosure.

Thus, the direct dyes may be chosen according to the logP value.

As non-limiting examples of dyes that are suitable for use in the composition of the present disclosure, mention may be made of the following dyes:

| STRUCTURE | NAME | logP |
|---|---|---|
| 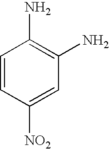 | 4-nitro-o-phenylenediamine | 0.88 |
| 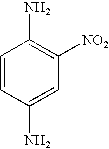 | 2-nitro-p-phenylenediamine | 0.53 |
| 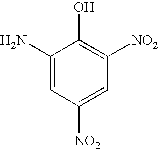 | picramic acid | 0.93 |
| 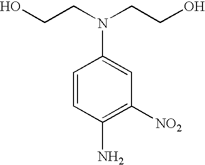 | HC Red 13 | 0.66 |
| 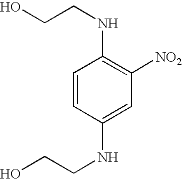 | N,N′-bis(2-hydroxyethyl)-2- nitro-p-phenylenediamine | −0.44 |
| 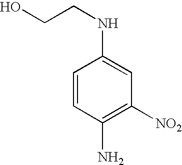 | HC Red 7 | 0.13 |
| 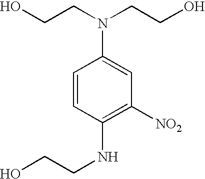 | HC Blue 2 | −0.32 |
| 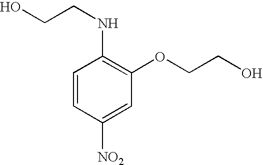 | HC Yellow 4 | 0.56 |
| 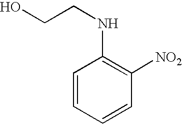 | HC Yellow 2 | 1.05 |
| 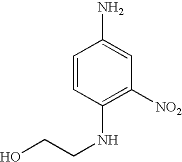 | HC Red 3 | −0.42 |
| 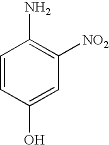 | 4-amino-3-nitrophenol | 1.19 |
| 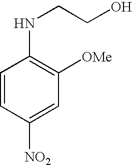 | 2-hydroxyethylamino-5- nitroanisole | 1.13 |
| 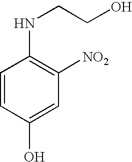 | 3-nitro-p- (hydroxyethylamino)phenol | 0.21 |
| 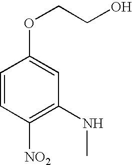 | 3-methylamino-4- nitrophenoxyethanol | 1.13 |
| 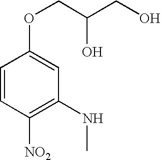 | 2-nitro-5-glyceryl-N- methylaniline | 0.89 |
| 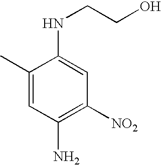 | HC Violet 1 | 0.67 |
| 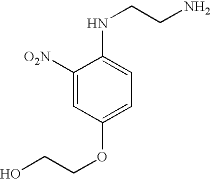 | HC Orange 2 | 0.15 |
| 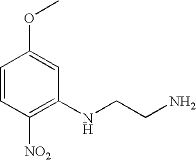 | HC Yellow 9 | 1.12 |
| 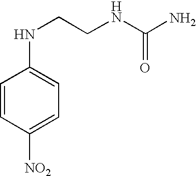 | 4-nitrophenylaminoethylurea | 0.59 |
| 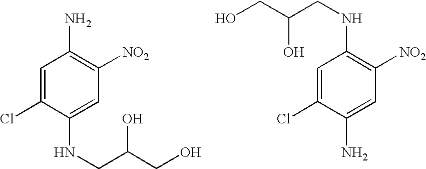 | HC Red 10 and HC Red 11 | 0.13 |
| 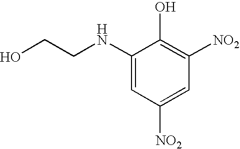 | (2-hydroxyethyl)picramic acid | 0.38 |
| 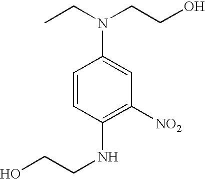 | HC Blue 12 | 1.15 |
| 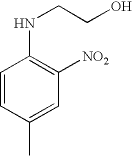 | 3-nitro-4-(N-(β- hydroxyethyl)amino)toluene | 1.59 |
| 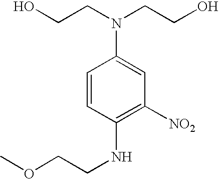 | 2-(N-(β-methoxyethyl)amino)-5- (N,N- bis(hydroxyethyl)amino)nitrobenzene | 0.38 |
| 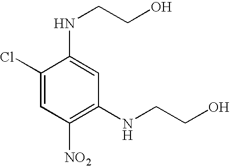 | HC Yellow 10 | 0.20 |
| 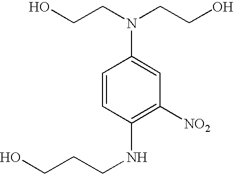 | HC Violet 2 | 0.17 |
| 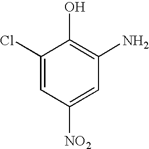 | 2-amino-6-chloro-4-nitrophenol | 1.53 |
| 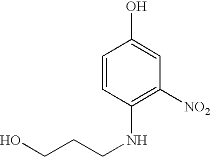 | 4-hydroxypropylamino-3- nitrophenol | 0.70 |
| 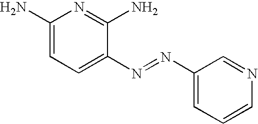 | 2,6-diamino-3-((pyridin-3- yl)azo)pyridine | 1.58 |
| 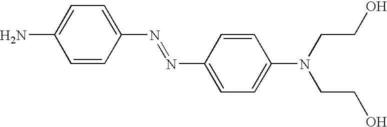 | Disperse Black 9 | 1.83 |
| 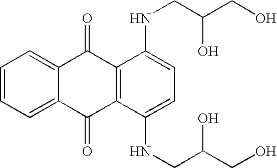 | HC Blue 14 | 0.62 |
| 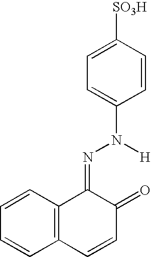 | Acid Orange 7 | 1.14 |
| 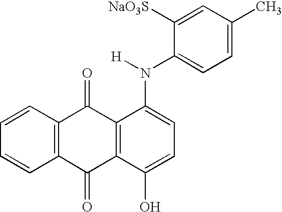 | Acid Violet 43 | 1 |
| 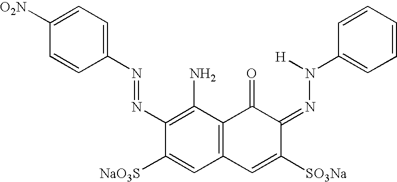 | Acid Black 1 | −4.53 |
| 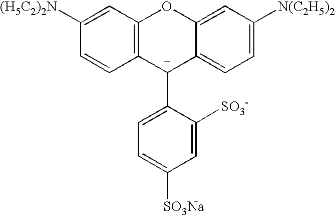 | Acid Red 52 | 1.3 |
| 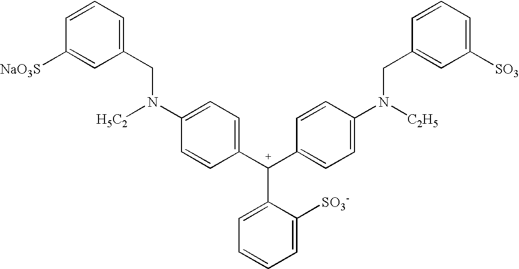 | Acid Blue 9 | −0.32 |
| 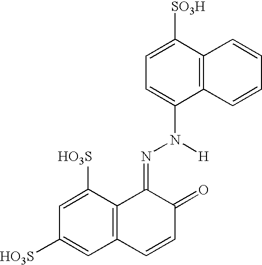 | Acid Red 18 | 1.63 |
| 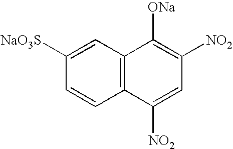 | Acid Yellow 1 | 1.22 |

The at least one direct dye exhibiting a logP value of less than or equal to 2 may be present in the composition in an amount ranging from 0.001 to 10% by weight, for example from 0.01 to 10% by weight, relative to the total weight of the composition.

The medium of the dyeing compositions of the present disclosure comprises at least one liquid organic solvent.

As used herein, the term “organic solvent” means an organic substance capable of dissolving another substance without chemically modifying it.

Organic solvents suitable for use in the present disclosure are chosen from compounds that are liquid at a temperature of 25° C. and at 105 Pa (760 mmHg) and which are different from the electrophilic monomers according to the invention.

As non-limiting examples of organic solvents that may be utilized in accordance with the present disclosure, mention may be made of: aromatic alcohols, including, but not limited to benzyl alcohol; liquid fatty alcohols, for example C₁₀-C₃₀ fatty alcohols; modified or unmodified polyols, for example glycerol, glycol, propylene glycol, dipropylene glycol, butylene glycol or butyl diglycol; volatile silicones, including, but not limited to cyclopentasiloxane, cyclohexasiloxane, polydimethylsiloxanes modified or unmodified by alkyl, amine, imine, fluoroalkyl, carboxyl, betaine, and/or quaternary ammonium functional groups; liquid modified polydimethylsiloxanes; mineral, organic or vegetable oils; alkanes, for example C₅ to C₁₀ alkanes; liquid fatty acids; and liquid fatty esters, for example liquid fatty alcohol benzoates or salicylates.

In a non-limiting embodiment of the present disclosure, the organic solvent is chosen from organic oils; silicones, for example volatile silicones, silicone gums or oils that are or are not aminated and mixtures thereof; mineral oils; vegetable oils, for example olive oil, castor oil, rapeseed oil, coconut oil, wheat germ oil, sweet almond oil, avocado oil, macadamia oil, apricot oil, safflower oil, candlenut oil, camelina oil, tamanu oil or lemon oil; and organic compounds, including, but not limited to C₅-C₁₀ alkanes, acetone, methyl ethyl ketone, liquid esters of C₁-C₂₀ acids and of C₁-C₈ alcohols, for example methyl acetate, butyl acetate, ethyl acetate and isopropyl myristate, dimethoxyethane, diethoxyethane, liquid C₁₀-C₃₀ fatty alcohols, for example oleyl alcohol, liquid C₁₀-C₃₀ fatty alcohol esters, for example C₁₀-C₃₀ fatty alcohol benzoates and mixtures thereof; polybutene oil, isononyl isononanoate, isostearyl malate, pentaerythrityl tetraisostearate, tridecyl trimelate, a mixture of cyclopentasiloxane (14.7% by weight)/polydimethylsiloxane dihydroxylated in the α and ω positions (85.3% by weight); and mixtures thereof.

In at least one embodiment of the present disclosure, the organic solvent comprises silicone or a silicone mixture, for example liquid polydimethylsiloxanes and liquid modified polydimethylsiloxanes, wherein the viscosity of the silicone and/or silicone mixture at 25° C. ranges from 0.1 cSt and 1 000 000 cSt, for example, from 1 cSt and 30 000 cSt.

Non-limiting mention may also be made of the following oils and mixtures of oils:

the α,ω-dihydroxylated polydimethylsiloxane/cyclopentadimethylsiloxane (14.7/85.3) mixture sold by Dow Corning under the name of DC 1501 Fluid;

the α,ω-dihydroxylated polydimethylsiloxane/polydimethylsiloxane mixture sold by Dow Corning under the name of DC 1503 Fluid;

the dimethicone/cyclopentadimethylsiloxane mixture sold by Dow Corning under the name of DC 1411 Fluid or that sold by Bayer under the name SF1214;

the cyclopentadimethylsiloxane sold by Dow Corning under the name of DC245 Fluid;

and mixtures thereof.

In addition to the at least one liquid organic solvent, the medium of the composition of the present disclosure may also comprise water. In at least one embodiment, the medium is anhydrous, meaning that the medium comprises less than 1% by weight of water, relative to the total weight of the composition.

The at least one organic solvent of the composition is present in an amount ranging from 0.01 to 99% by weight, for example, from 50% to 99% by weight, relative to the total weight of the composition.

The composition of the present disclosure may be encapsulated and/or provided in the form of an emulsion, wherein the electrophilic monomers are held in an anhydrous medium until the moment of use. When the composition of the present disclosure is in the form of an emulsion, this emulsion is composed, for example, of a dispersed or continuous phase, which can comprise water, C₁-C₄ aliphatic alcohols or their mixtures, and an anhydrous organic phase comprising the monomer. When the composition of the present disclosure is encapsulated in capsules or microcapsules, the capsules or microcapsules may comprise the electrophilic monomer in an anhydrous medium and may be dispersed in an anhydrous medium as defined above, water, C₁-C₄ aliphatic alcohols and mixtures thereof.

The compositions of the present disclosure may further comprise at least one polymerization inhibitor to increase the stability of the composition over time. Non-limiting examples of such polymerization inhibitors include anionic and/or radical polymerization inhibitors. Mention may be made, in a non-limiting way, of the following polymerization inhibitors: sulphur dioxide, nitric oxide, lactone, boron trifluoride, hydroquinone and derivatives thereof, for example hydroquinone monoethyl ether or tert-butylhydroquinone, benzoquinone and derivatives thereof, for example duroquinone, catechol and derivatives thereof, for example t-butylcatechol and methoxycatechol, anisole and derivatives thereof, for example methoxyanisole or hydroxyanisole, pyrogallol and derivatives thereof, p-methoxyphenol, hydroxybutyltoluene, alkyl sulphates, alkyl sulphites, alkyl sulphones, alkyl sulphoxides, alkyl sulphides, mercaptans, and mixtures thereof. In at least one embodiment of the present invention, the alkyl groups of the aforementioned polymerization inhibitors comprise 1 to 6 carbon atoms.

The polymerization inhibitor may be present in the composition according to the present disclosure in an amount ranging from 10 ppm to 30%, for example from 10 ppm to 15% by weight, relative to the total weight of the composition.

As non-limiting examples of other inhibitors that may be utilized in the compositions of the present disclosure, non-limiting mention may be made of inorganic or organic acids.

In at least one embodiment of the present disclosure, the acid inhibitor is chosen from inorganic acids and organic acids having one or more carboxyl or sulpho groups and exhibiting a pKa ranging from 0 to 6.

As non-limiting examples of such acids, mention may be made of phosphoric acid, hydrochloric acid, nitric acid, sulphonic acid, for example benzene- or toluenesulphonic acid, sulphuric acid, carbonic acid, hydrofluoric acid, acetic acid, formic acid, propionic acid, octanoic acid, heptanoic acid, hexanoic acid, benzoic acid, mono-, di- or trichloroacetic acid, salicylic acid and trifluoroacetic acid.

In at least one embodiment of the present disclosure, the acid is chosen from organic acids. In a further embodiment, the organic acid is acetic acid.

When it is present, the acid is present in the composition in an amount ranging from 0.01% to 30%, for example, from 0.01% to 15% by weight, relative to the total weight of the composition.

The compositions of the present disclosure may further comprise at least one conventional pigment, and may also comprise metal powders or particles, including, for example, powders or particles formed of aluminium, zinc, copper, and the like.

The compositions of the present disclosure may further comprise known cosmetic active agents. As non-limiting examples of such agents, mention may be made of fillers, polymers, elastomers, fixing or non-fixing polymers, conditioning polymers, organic or inorganic pigments, pearlescent agents, direct dyes other than the hydrophilic dyes of the present disclosure, oxidation dyes, reducing agents, oxidizing agents, fatty substances, silicones, thickening agents, softening agents, antifoaming agents, moisturizing agents, emollients, basifying agents, plasticizers, sunscreens, clays, colloidal minerals, fragrances, peptizing agents, preservatives, anionic, cationic, amphoteric, zwitterionic or nonionic surfactants, proteins, vitamins, and the like.

In order to improve the adhesion of the polymer formed in situ by reaction of the electrophilic monomers according to the invention to a keratinous fiber, the fiber can be pretreated with a polymer.

The compositions of the present disclosure may be provided in various forms, including, but not limited to lotions, sprays or foams, and may be applied in the form of a shampoo or conditioner.

When the compositions of the present disclosure are in the form of a spray, the compositions may further comprise a propellant. The propellant is composed of compressed or liquefied gases conventionally used in the preparation of aerosol compositions. As non-limiting examples of such gases, mention may be made of air, carbon dioxide gas, compressed nitrogen and soluble gases, for example dimethyl ether, halogenated (,e.g., fluorinated) or non-halogenated hydrocarbons and mixtures thereof.

As mentioned above, the present disclosure also relates to a process for dyeing keratinous substances. In at least one embodiment, this process comprises applying a composition according to the present disclosure to a keratinous substance, for example keratinous fibers such as the hair, wherein the composition according to the present disclosure comprises at least one electrophilic monomer and at least one hydrophilic dye having a logP of less than or equal to 2.

In at least one embodiment, the composition of the present disclosure is applied to a keratinous substance, such as keratinous fibers, e.g., hair, in the presence of a nucleophilic agent.

In at least one embodiment of the present disclosure, the nucleophilic agent capable of initiating the polymerization of the cyanoacrylate monomer can be applied beforehand to keratinous fibers. The nucleophilic agent may be pure, in solution, in the form of an emulsion, or encapsulated. If the composition of the present disclosure is anhydrous, the nucleophilic agent may be added to an anhydrous composition at the time of use, i.e., immediately before application to keratinous fibers.

In a further embodiment of the present disclosure, the nucleophilic agent is water. In this case, the nucleophilic agent may be introduced, for example, by preliminary moistening of the keratinous fibers. It may also be added directly to the composition before application to the fibers.

In another embodiment of the present disclosure, the kinetics of polymerization may be adjusted by moistening the keratinous material beforehand using an aqueous solution, the pH of which has been adjusted using a base, an acid or an acid/base mixture. Non-limiting examples of such acids and/or bases include inorganic or organic acids and bases.

In another non-limiting embodiment of the present disclosure, the process of the present disclosure may be carried out in multiple stages: a first stage comprising applying a composition comprising the at least one hydrophilic dye having a logP of less than or equal to 2 to keratinous fibers; and a second stage comprising applying a composition comprising at least one electrophilic monomer to the fibers. In this embodiment, the nucleophilic agent is present in a separate composition. In addition, intermediate rinsing or other treatments may be applied. The aforementioned two stages may also be separated in time.

The process of the present disclosure may further comprise intermediate or final stages, such as the application of a cosmetic product, a rinsing stage or a drying stage. Drying may be carried out, for example, with a hood dryer, with a hand-held hairdryer and/or with a smoothing iron. In one embodiment, application of a composition in accordance with the present disclosure is followed by a rinsing operation.

The composition of the present disclosure may also be applied multiple times to the same keratinous material in order to obtain a superimposition of layers and achieve a deposited material having desired chemical and mechanical properties, thickness, appearance and/or feel.

In order to improve, inter alia, the adhesion of the poly(cyanoacrylate) polymer formed in situ upon application of a composition according to the present disclosure to keratin fibers, the keratin fibers may be pretreated with any type of polymer.

In order to adjust the kinetics of anionic polymerization, it is also possible to increase the nucleophilicity of the keratinous fiber by chemical conversion thereof. Mention may be made, by way of example, of the reduction of the disulphide bridges of which the keratin is partially composed to produce thiols before application of the composition of the invention. Mention may also be made, as non-limiting examples, as reducing agents suitable for reducing the disulphide bridges of which the keratin is partially composed, of the following compounds: anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formaldehyde sulphoxylate, isooctyl thioglycolate, d,l-cysteine or monoethanolamine thioglycolate.

The application of the composition of the present disclosure may also be preceded by a hair treatment, such as, for example, direct or oxidation dyeing.

In at least one embodiment of the present disclosure, the at least one electrophilic monomer is chosen from monomers capable of polymerizing on keratinous fibers under cosmetically acceptable conditions. In yet another non-limiting embodiment, the polymerization of the at least one electrophilic monomer is carried out at a temperature of less than or equal to 80° C., which does not prevent the application being terminated by drying with a hood dryer, blow drying, and contact with a flat iron or curling tong.

The present disclosure further relates to a multi-compartment dyeing kit comprising, in one compartment, a first composition comprising the at least one electrophilic monomer and at least one hydrophilic direct dye and, in another compartment, a second composition comprising at least one nucleophilic agent, wherein either of the compartments may comprise at least one liquid organic solvent.

In at least one non-limiting embodiment, the liquid organic solvent is present in the same compartment as the at least one electrophilic monomer. According to this embodiment, the composition comprising the at least one hydrophilic direct dye and the at least one electrophilic monomer may be anhydrous.

In another embodiment, the present disclosure relates to a multi-compartment dyeing kit comprising, in one compartment, a first composition which comprises the at least one electrophilic monomer and, in another compartment, a second composition which comprises at least one hydrophilic direct dye, either comprising at least one liquid organic solvent, in one embodiment with the at least one electrophilic monomer. According to this embodiment, the composition comprising the at least one electrophilic monomer can be an anhydrous composition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature.

In the examples, the percentages are expressed as weight percentages of active material, except as otherwise specifically noted.

### EXAMPLES

Tests were carried out using the following compositions:

1-HC Red 13

The following composition was prepared:

| | | |
|---|---|---|
| DC 1501 Fluid | 40 | g |
| DC 245 Fluid | 39 | g |
| HG Red 13 (logP = 0.66) | 1 | g |
| Methylheptyl cyanoacrylate from Chemence | 10 | g |
| Acetic acid* | 0.25 | g |

0.5 g of the composition was applied to a lock of 1 g of clean and wet hair. After a leave-in time of 15 minutes, the lock was dried with a hair dryer for 2 minutes. The lock obtained was colored red and was resistant to shampooing operations.

2-HC Blue 14

The following composition was prepared:

| | | |
|---|---|---|
| DC 1501 Fluid | 40 | g |
| DC 245 Fluid | 39 | g |
| HC Blue 14 (logP = 0.62) | 1 | g |
| Methylheptyl cyanoacrylate from Chemence | 10 | g |
| Acetic acid* | 0.25 | g |

0.5 g of the composition was applied to a lock of 1 g of clean and wet hair. After a leave-in time of 15 minutes, the lock was dried with a hair dryer for 2 minutes. The lock obtained was colored blue and was resistant to shampooing operations.

3-Disperse Black 9

The following composition was prepared:

| | | |
|---|---|---|
| DC 1501 Fluid | 40 | g |
| DC 245 Fluid | 39.8 | g |
| Disperse Black 9 (logP = 1.83) | 0.2 | g |
| Methylheptyl cyanoacrylate from Chemence | 10 | g |
| Acetic acid* | 0.25 | g |

0.5 g of the composition was applied to a lock of 1 g of clean and wet hair. After a leave-in time of 15 minutes, the lock was dried with a hair dryer for 2 minutes. The lock obtained was colored black and was resistant to shampooing operations.

4-HC Yellow 10

The following composition was prepared:

| | | |
|---|---|---|
| DC 1501 Fluid | 40 | g |
| DC 245 Fluid | 39.5 | g |
| HC Yellow 10 (logP = 0.20) | 0.5 | g |
| Methylheptyl cyanoacrylate from Chemence | 10 | g |
| Acetic acid* | 0.25 | g |

0.5 g of the composition was applied to a lock of 1 g of clean and wet hair. After for a leave-in time of 15 minutes, the lock was dried with a hair dryer for 2 minutes. The lock obtained was colored yellow and was resistant to shampooing operations.

5-Mixture of Acid Dyes

The following dyeing formulation “A” was prepared:

Composition of formulation “A”

| | C (g %) |
|---|---|
| Denatured alcohol | 22.34 |
| Benzyl alcohol | 10 |
| Glycerol | 5 |
| Citric acid | 4 |
| Lactic acid | 1.98 |
| Hydroxyethylcellulose | 1.20 |
| Xanthan gum | 0.40 |
| Sodium PCA | 0.25 |
| Hydroxypropyltrimonium | 0.03 |
| hydrolysed wheat protein | |
| Phenoxyethanol | 0.0002 |
| Acid Orange 7 (logP = 1.14) | 0.1962 |
| Acid Violet 43 (logP = 1) | 0.0556 |
| Acid Black 1 (logP = 4.53) | 0.0243 |
| Acid Red 52 (logP = 1.3) | 0.0297 |
| Acid Blue 9 (logP = 0.01) | 0.01 |
| Acid Red 18 (logP = 1.63) | 0.0087 |
| Acid Yellow 1 (logP = 1.22) | 0.0056 |
| Water | q.s. for 100 |

At the time of use, 10 g of the formulation “A” and 1 g of octyl cyanoacrylates were mixed in a plastic container, resulting in mixture “B”. Mixture “B” was subsequently applied to 1 g of a lock of hair comprising 90% of natural white hairs.

The leave-in time was 30 min at ambient temperature. At the end of the 30 min, the lock is rinsed.

The lock was then subjected to a series of 6 and then 12 shampooing operations. For each shampooing cycle, the following protocol was used

1. application to the lock of 0.4 g of Elseve Multivitamin shampoo and massaging for 10 seconds;

2. leaving for 5 min and then rinsing with water for 10 seconds;

3. drying with a hood hairdryer.

The color of the locks before and after the washing operations was evaluated in the L*a*b* system using a spectrophotometer, Minolta® CM 2002 colorimeter, illuminant D65-10° CSI.

In the L*a*b* system, L* represents the intensity of the color, a* indicates the green/red color axis and b* indicates the blue/yellow color axis.

The lower the value of L, the darker the color or the more intense it is. The higher the value of a*, the redder the hue and, the higher the value of b*, the bluer the hue.

The loss in color ΔE after shampooing was determined according to the following equation:
ΔE=\sqrt{(L₁−L₀)²+(a₁*−a₀*)²+(b₁*−b₀*)²}

In this equation, L₁, a₁* and b₁* represent the calorimetric values of the locks measured after shampooing operations and L₀, a₀* and b₀* represent the values measured before shampooing.

The greater the value of ΔE, the greater the difference in color before and after washing operations and the less resistant the coloring is to shampooing operations.

At the same time, a control was evaluated. In this control, 10 g of dyeing formulation “A” was applied to 1 g of a lock of hair comprising 90% of natural white hairs. The application and measurement conditions were the same as those described above with the mixture “B”.

Results Obtained
Mixture “B” did not adhere to the plastic container or to gloves, in contrast to the formulation “A”.

As shown in the table below, the lock colored with mixture “B” was more resistant to shampooing than the lock treated solely with the formulation “A”.

| | | Loss (ΔF) after |
|---|---|---|
| | Loss (ΔE) after 6 | 12 shampooing |
| | shampooing operations | operations |
| Formulation | 7.59 | 12.60 |
| “A” | | |
| Formulation | 3.48 | 6.19 |
| “B” | | |

What is claimed is:

1. A dyeing composition comprising, in a medium suitable for dyeing, at least one electrophilic monomer, at least one hydrophilic direct dye having a logP of less than or equal to 2 and at least one liquid organic solvent.

2. The composition of claim 1, wherein the at least one electrophilic monomer is chosen from monomers of formula (A): 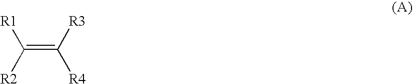
wherein:
R1 and R2 are each independently a group having little to no electron-withdrawing effect; and
R3 and R4 are each independently an electron-withdrawing group.

3. The composition of claim 2, wherein R1 and R2 are independently chosen from:
hydrogen;
saturated or unsaturated, linear, branched or cyclic hydrocarbon groups optionally comprising at least one atom chosen from nitrogen, oxygen and sulphur atoms and optionally substituted by at least one group chosen from —OR, —COOR, —COR, —SR and halogen atoms, R being chosen from a hydrogen atom or a saturated or unsaturated, linear, branched or cyclic hydrocarbon group comprising from 1 to 20 carbon atoms, optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, R′ being chosen from C₁-C₁₀ alkyl groups;
modified or unmodified polyorganosiloxane residues; and
polyoxyalkylene groups.

4. The composition of claim 3, wherein at least one of R1 and R2 is chosen from saturated or unsaturated, linear, branched or cyclic C₁-C₂₀ hydrocarbon groups.

5. The composition of claim 3, wherein said at least one electrophilic monomer is chosen from cyanoacrylate monomers of formula (B): 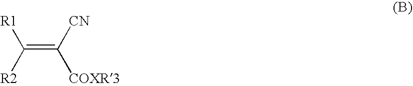
wherein:
X is chosen from NH, S and O
R′3 is chosen from a hydrogen atom and R; and
R1 and R2 are independently chosen from:
hydrogen
saturated or unsaturated, linear, branched or cyclic hydrocarbon groups optionally comprising at least one atom chosen from nitrogen, oxygen and sulphur atoms and optionally substituted by at least one group chosen from —OR, —COOR, —COR, —SR and halogen atoms, R being chosen from a hydrogen atom and a saturated or unsaturated, linear, branched or cyclic hydrocarbon group comprising from 1 to 20 carbon atoms, optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, R′ being chosen from C₁-C₁₀ alkyl groups;
modified or unmodified polyorganosiloxane residues; and
polyoxyalkylene groups.

6. The composition of claim 5, wherein said cyanoacrylate monomers are chosen from those of formula (C); 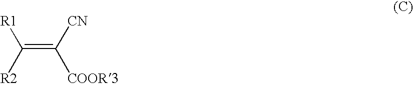
wherein
R′3 is chosen from C₁-C₁₀ alkyl, C₂-C₁₀ alkenyl and (C₁-C₄)alkoxy(C₁-C₁₀)alkyl radicals and
R1 and R2 are independently chosen from:
hydrogen
saturated or unsaturated, linear, branched or cyclic hydrocarbon groups optionally comprising at least one atom chosen from nitrogen, oxygen and sulphur atoms and optionally substituted by at least one group chosen from —OR, —COOR, —COR, —SR and halogen atoms, R being chosen from a hydrogen atom and a saturated or unsaturated, linear, branched or cyclic hydrocarbon group comprising from 1 to 20 carbon atoms, optionally comprising at least one atom chosen from nitrogen, oxygen and sulphur atoms and optionally substituted by at least one group chosen from —OR′, —COOR′, —COR′, —SH, —SR′, —OH, halogen atoms, and a residue of a polymer, R′ being chosen from C₁-C₁₀ alkyl groups;
modified or unmodified polyorganosiloxane residues; and
polyoxyalkylene groups.

7. The composition of claim 1, wherein the electrophilic monomer is an alkyl cyanoacrylate of formula (F): 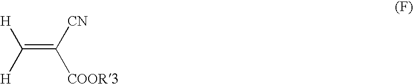
wherein R′3 is chosen from: —(CH₂)₇—CH₃, —CH(CH₃)—(CH₂)₅—CH₃, —CH₂—CH(C₂H₅)—(CH₂)₃—, CH₃, —(CH₂)₅—CH(CH₃)—CH₃, and —(CH₂)₄—CH(C₂H₅)—CH₃.

8. The composition of claim 1, wherein the at least one electrophilic monomer is present in an amount ranging from 0.1 to 80% by weight, relative to the total weight of the composition.

9. The composition of claim 1, wherein the at least one hydrophilic direct dye having a logP of less than or equal to 2 is chosen from neutral, acidic or cationic nitrobenzene direct dyes, neutral, acidic or cationic azo direct dyes, quinone direct dyes, azine direct dyes, triarylmethane direct dyes, indoamine direct dyes and natural direct dyes.

10. The composition of claim 9, wherein the at least one hydrophilic direct dye is chosen from 4-nitro-o-phenylenediamine, 2-nitro-p-phenylenediamine, picramic acid, HC Red 13, N,N′-bis(2-hydroxyethyl)-2-nitro-p-phenylenediamine, HC Red 7, HC Blue 2, HC Yellow 4, HC Red 3, 4-amino-3-nitrophenol, 1-hydroxyethylamino-5-nitroanisole, 3-nitro-p-(hydroxyethylamino)phenol, 3-methylamino-4-nitrophenoxyethanol, 2-nitro-5-glyceryl-N-methylaniline, HC Violet 1, HC Orange 2, HC Yellow 9, 4-nitrophenylaminoethylurea, (2-hydroxyethyl)picramic acid, HC Blue 12, 3-nitro-4-(N-(β-hydroxyethyl)amino)toluene, 2-(N-(β-methoxyethyl)amino)-5-(N,N-bis(hydroxyethyl)amino)nitrobenzene, HC Yellow 10, HC Violet 2, 2-amino-6-chloro-4-nitrophenol, 4-hydroxypropylamino-3-nitrophenol, HC Yellow 13, 2,6-diamino-3-((pyridin-3-yl)azo)pyridine, Disperse Black 9, HC Blue 14, HC Red 10, HC Red 11, Acid Orange 7, Acid Violet 43, Acid Black 1, Acid Red 52, Acid Blue 9, Acid Red 18 and Acid Yellow 1.

11. The composition of claim 1, where the at least one hydrophilic direct dye is present in an amount ranging from 0.001 to 10% by weight, relative to the total weight of the composition.

12. The composition of claim 1, wherein the medium suitable for dyeing comprises less than 1% by weight of water, relative to the total weight of the composition.

13. The composition of claim 1, further comprising at least one nucleophilic agent.

14. A process for dyeing keratinous substances, comprising applying, in the presence of a nucleophilic agent, to said keratinous substances a dyeing composition comprising, in a medium suitable for dyeing, at least one electrophilic monomer, at least one hydrophilic direct dye having a logP of less than or equal to 2 and at least one liquid organic solvent.

15. The process of claim 14, wherein said keratin fibers are human hair.

16. A dyeing kit comprising at least two compartments, wherein one compartment comprises a composition comprising at least one hydrophilic direct dye having a logP of less than or equal to 2 and at least one electrophilic monomer and another compartment comprises a second composition comprising a nucleophilic agent, either compartment optionally comprising at least one liquid organic solvent.

17. A dyeing kit comprising at least two compartments, wherein one compartment comprises a composition comprising at least one electrophilic monomer and another compartment comprises a second composition comprising at least one direct dye having a logP of less than or equal to 2, either compartment optionally comprising at least one liquid organic solvent.
